(19) United States
(12) Patent Application Publication
(10) Pub. No.: US 2013/0136720 A1
(43) Pub. Date: May 30, 2013

(54) METHODS OF USING FUT2 GENETIC VARIANTS TO DIAGNOSE CROHN'S DISEASE

(71) Applicants: Dermot P. McGovern, Los Angeles, CA (US); Jerome I. Rotter, Los Angeles, CA (US); Stephan R. Targan, Santa Monica, CA (US); Kent D. Taylor, Ventura, CA (US); Xiuqing Guo, Santa Monica, CA (US)

(72) Inventors: Dermot P. McGovern, Los Angeles, CA (US); Jerome I. Rotter, Los Angeles, CA (US); Stephan R. Targan, Santa Monica, CA (US); Kent D. Taylor, Ventura, CA (US); Xiuqing Guo, Santa Monica, CA (US)

(73) Assignee: CEDARS-SINAI MEDICAL CENTER, Los Angeles, CA (US)

(21) Appl. No.: 13/521,622

(22) PCT Filed: Jan. 14, 2011
(86) PCT No.: PCT/US11/21382

§ 371 Date: Jul. 11, 2012

Related U.S. Application Data
(60) Provisional application No. 61/295,309, filed on Jan. 15, 2010.

(51) Int. Cl. C12Q 1/68

(52) U.S. Cl. CPC C12Q 1/6883
USPC 424/93.4; 506/2

(57) ABSTRACT

The present invention relates to prognosing, diagnosing and treating of Crohn's disease. The invention also provides prognosis, diagnosis, and treatment that are based upon the presence of one or more genetic risk factors at the FUT2 genetic locus.

### CROSS-REFERENCE TO RELATED APPLICATIONS

The present application claims the benefit of the filing date of U.S. Provisional Application No. 61/295,309 filed Jan. 15, 2010, the disclosure of which is incorporated herein by reference in its entirety.

### GOVERNMENT RIGHTS

This invention was made with government support under NCRR grant MOI-RR00425 and NIH grant P01-DK046763. The government has certain rights in the invention.

### FIELD OF INVENTION

The invention relates generally to the field of inflammatory disease, specifically to Crohn's disease.

### BACKGROUND

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (61, 62). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (62). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (63). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (12, 63, 64, 65, 66). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

In one embodiment, the invention provides a method of diagnosing susceptibility to Crohn's disease in an individual, comprising: obtaining a sample from the individual, assaying the sample to determine the presence or absence of a risk variant at the FUT2 genetic locus, and diagnosing susceptibility to Crohn's disease in the individual based on the presence of the risk variant at the FUT2 genetic locus. The risk variant can be selected from the group consisting of rs602662, rs676388, rs485186, and rs504963. Assaying of the sample comprises genotyping for one or more single nucleotide polymorphisms. The sample can be whole blood, plasma, serum, saliva, cheek swab, urine, or stool.

In another embodiment, the invention provides a method of determining a high probability of developing Crohn's disease in an individual, relative to a healthy subject, comprising: obtaining a sample from the individual, assaying the sample to determine the presence or absence of one or more risk variants at the FUT2 genetic locus, and diagnosing a high probability of developing Crohn's disease in the individual, relative to a healthy subject, based upon the presence of one or more risk variants at the FUT2 genetic locus. The risk variant can be selected from the group consisting of rs602662, rs676388, rs485186, and rs504963 Assaying of the sample comprises genotyping for one or more single nucleotide polymorphisms. The sample can be whole blood, plasma, serum, saliva, cheek swab, urine, or stool.

In a related embodiment, the invention provides a method of prognosing Crohn's disease in an individual, comprising: obtaining a sample from the individual, assaying the sample for the presence or absence of one or more genetic risk variants, and prognosing an aggressive form of Crohn's disease based on the presence of one or more risk variants at the FUT2 genetic locus. The risk variant can be selected from the group consisting of rs602662, rs676388, rs485186, and rs504963. Assaying of the sample comprises genotyping for one or more single nucleotide polymorphisms. The sample can be whole blood, plasma, serum, saliva, cheek swab, urine, or stool.

In a further embodiment, the invention provides method of treating an individual for Crohn's disease, comprising: prognosing an aggressive form of Crohn's disease in the individual based on the presence of one or more risk variants at the FUT2 genetic locus, and treating the individual, wherein the one or more risk variants are selected from rs602662, rs676388, rs485186, and rs504963. Assaying the sample comprises genotyping for one or more single nucleotide polymorphisms. The sample can be whole blood, plasma, serum, saliva, cheek swab, urine, or stool.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1. Graphical representation of an association between FUT2and CD. Circles—The GWAS population. Squares—The independent case-control replication cohort.

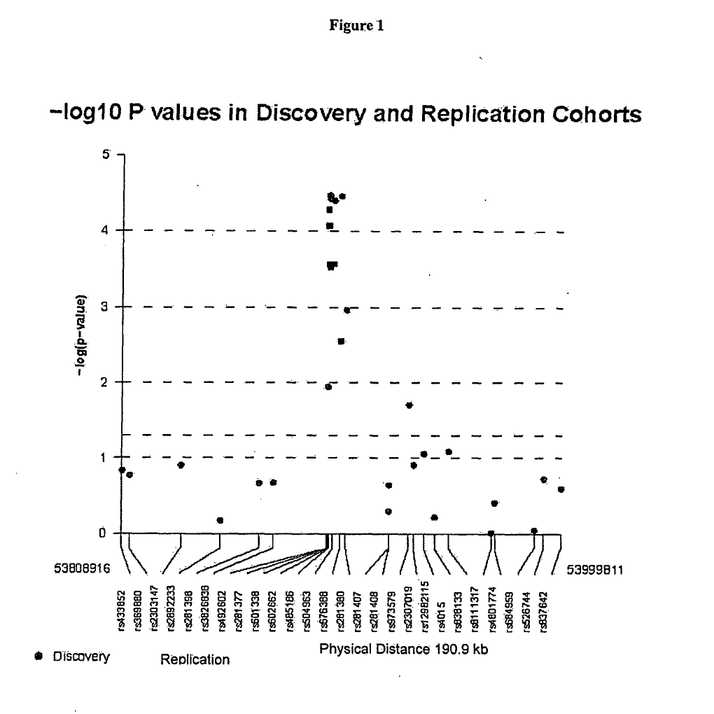

FIG. 2. Principal Component Plot for components 1 (C1—y axis) and 2 (C2—x axis) in CD and controls. The circled cases and controls are on the ‘Caucasian’ axis and were included in logistic regression analysis.

FIG. 3. Table listing the replication of confirmed and ‘nominally associated’ CD susceptibility loci from CD GWAS meta-analysis¹³. Chr.—chromosome

FIG. 4. Table listing novel loci associated with CD (cut off p=<1.0×10⁴). Chr.—chromosome.

FIG. 5. Table summarizing the association between FUT2 and CD in GWAS, confirmatory cohort of 1174 cases and 357 controls and the p value for association by the CD GWAS meta-analysis from Barrett et al. *P value calculated using logistic regression. **Combined p value calculated for p value in original GWAS and one tailed p value in independent replication. Synon.—synonymous.

FIG. 6. Graphical representation of the linkage disequilibrium and haplotype structure across the 6 FUT2 SNPs. Figure and data generated in HAPLOVIEW. Figures represent the LD in percent between SNPs as represented by D′.

### DESCRIPTION OF THE INVENTION

Crohn's disease (CD), one of the major forms inflammatory bowel diseases (IBD), is a chronic, debilitating disease characterized by recurrent gastrointestinal inflammation, postulated to occur as a result of an abnormal immune reaction to commensal flora in genetically susceptible individuals. The role of commensal flora in potentiating chronic gastrointestinal mucosal inflammation is substantiated by data from established rodent models of IBD such as the Il10^{-/-} mouse and the Hla-B27 transgenic rat that are disease free when kept in germ free environments but develop inflammation when raised under pathogen free conditions (1,2) Furthermore, in both of these models, the bacterial load and the nature of the commensal flora can influence either the site or degree of gastrointestinal inflammation (1,3,4). In human disease, antibiotic and probiotic therapy can be effective in modifying some of the manifestations of IBD (5,6).

Through utilizing genome-wide association studies (GWAS), in addition to candidate gene approaches, considerable success has been achieved in identifying genetic loci that increase susceptibility to CD in populations of Northern European origin (7-12). To date more than thirty loci are definitively known to be associated with CD, although these loci only account for a minority of the genetic variance to CD in the Caucasian population (13). A number of the CD susceptibility genes encode important components of the innate immune system genes such as NOD2 (11,12). The Toll like receptors (14,15) and the autophagy genes ATG16L1 and IRGM, emphasizing the importance of the microbial-host interaction in the development of CD. Furthermore, antibodies to bacterial antigens have been identified that define certain sub-groups of CD patients, reinforcing the essential role that bacteria play in driving CD (16).

As disclosed herein, a CD genome-wide association study (GWAS) was performed by the inventors, identifying a number of novel associations with CD. Considering the importance of the host-microbial interaction, the novel association with Fucosyltransferase 2 (FUT2), also termed secretor factor (Se), was of particular interest. FUT2 is a physiological trait that regulates the expression of the H antigen, a precursor of the blood group A and B antigens, on the gastrointestinal mucosa. Approximately 20% of Caucasians are non-secretors who do not express ABO antigens in saliva as they are homozygous for FUT2 null alleles (17). Genetic variation in FUT2 has been implicated in susceptibility to Helicobacter pylori infection (18), Noroviruses (Norwalk virus) (19-21), and progression of HIV (22). FUT2 alleles have also been associated with circulating serum vitamin B12 levels (23). Furthermore non-secretion of ABO blood group antigens into body fluids has been shown to be associated with the development of oral candidiasis (24,25), rheumatic fever (26), recurrent urinary tract infection (27), cholera (28) and infection with meningococcus (29), pneumococcus (29), and haemophilus influenzae (30). The data presented herein indicate an association between the non-secretor status associated FUT2 genotype and CD.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “inflammatory bowel disease” or “IBD” refers to gastrointestinal disorders including, but not limited to Crohn's disease (CD), ulcerative colitis (UC), and indeterminate colitis (IC). Inflammatory bowel diseases such as CD, UC, and IC are distinguished from all other disorders, syndromes, and abnormalities of the gastroenterological tract, including irritable bowel syndrome (IBS).

“Risk variant” as used herein refers to genetic variants, the presence of which correlates with an increase or decrease in susceptibility to Crohn's disease. Risk variants of Crohn's disease include, but are not limited to variants at the FUT2 genetic locus, such as “haplotypes” and/or a set of single nucleotide polymorphisms (SNPs) on a gene or chromatid that are statistically associated. More preferably, risk variants can include, but are not limited to rs602662, rs676388, rs485186, and rs504963.

“Treatment” or “treating,” as used herein refer to both therapeutic treatment and prophylactic or preventative measures, wherein the object is to prevent, slow down and/or lessen the disease even if the treatment is ultimately unsuccessful. Those in need of treatment include those already with Crohn's disease as well as those prone to have Crohn's disease or those in whom Crohn's disease is to be prevented. For example, in Crohn's disease treatment, a therapeutic agent may directly decrease the pathology of IBD, or render the cells of the gastroenterological tract more susceptible to treatment by other therapeutic agents.

As used herein, “diagnose” or “diagnosis” refers to determining the nature or the identity of a condition or disease. A diagnosis may be accompanied by a determination as to the severity of the disease. Diagnosis as it relates to the present invention, relates to the diagnosis of Crohn's disease.

As used herein, “prognostic” or “prognosis” refers to predicting the probable course and outcome of IBD or the likelihood of recovery from IBD. The prognosis can include the presence, the outcome, or the aggressiveness of the disease.

As used herein, the term “biological sample” or “sample” means any biological material obtained from an individual from which nucleic acid molecules can be prepared. Examples of a biological sample include, but are not limited to whole blood, plasma, serum, saliva, cheek swab, urine, stool, or other bodily fluid or tissue that contains nucleic acid.

In one embodiment, the present invention provides a method of diagnosing susceptibility to Crohn's Disease in an individual, relative to a healthy individual, by determining the presence or absence of a risk variant at the FUT2 genetic locus, where the presence of the risk variant at the FUT2 genetic locus is indicative of susceptibility to Crohn's Disease in the individual. In another embodiment, the risk variant comprises the SNP rs602662, rs676388, rs485186, or rs504963. In one embodiment, the risk variant can be at loci including, but are not limited to ASHL, ARPC1A, RHOU, RBP1 and 2, TACR3, MMD2, NPSR1, ACER2, AP3D1, or SPG20.

In one embodiment, the present invention provides a method of treating Crohn's Disease by determining the presence of a risk variant at the FUT2 genetic locus and treating the individual. The risk variant comprises the SNP rs602662, rs676388, rs485186, and rs504963. In one embodiment, the one or more risk variants can be at loci including, but are not limited to ASHL, ARPC1A, RHOU, RBP1 and 2, TACR3, MMD2, NPSR1, ACER2, AP3D1, or SPG20.

In another embodiment, the present invention provides a method of prognosing Crohn's Disease by determining the presence or absence of one or more risk variants at the FUT2 genetic locus and prognosing a complicated form of Crohn's Disease based on the presence of the one or more risk variants at the FUT2 genetic locus. The risk variant comprises the SNP rs602662, rs676388, rs485186, and rs504963. In one embodiment, the one or more risk variants can be at loci including, but are not limited to ASHL, ARPC1A, RHOU, RBP1 and 2, TACR3, MMD2, NPSR1, ACER2, AP3D1, or SPG20.

In one embodiment, the present invention provides a method of diagnosing a high probability of developing Crohn's Disease in an individual, relative to a healthy individual, by determining the presence or absence of one or more risk variants at the FUT2 genetic locus, where the presence of the one or more risk variants at the FUT2 genetic locus is indicative of a low probability of developing Crohn's Disease in an individual. The risk variant comprises the SNP rs602662, rs676388, rs485186, and rs504963. In one embodiment, the one or more risk variants can be at loci including, but are not limited to ASHL, ARPC1A, RHOU, RBP1 and 2, TACR3, MMD2, NPSR1, ACER2, AP3D1, or SPG20.

In another embodiment, an individual with Crohn's disease having one or more genetic risk variants at CD associated loci specifically involved in the host-microbial interaction, exemplified by, but not limited to, SPG20 and FUT2, is treated by antibiotic and or probiotic based treatment therapies. In yet another embodiment, the antibiotic and probiotic treatments are administered as a preventative measure to individuals who have been identified as having a higher than normal risk of developing CD, based upon the presence of one or more genetic variants at CD associated loci specifically involved in the host-microbial interaction, exemplified by, but not limited to, SPG20 and FUT2.

In another embodiment, the present invention provides a method of prognosing Crohn's Disease by determining the presence or absence of one or more risk variants of genetic loci at SPG20 and FUT2, and prognosing pathogenesis, mediated in whole or in part by host-microbial interaction, based on the presence of the one or more risk variants at one or more of SPG20 and FUT2 genetic loci.

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (69).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., (67). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (68, 73). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (69). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (70, 71).

The technique of single strand conformational, polymorphism (SSCP) also may be used to detect the presence or absence of a SNP and/or a haplotype (72). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (73).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (74). Furthermore, one skilled in the art understands that, where the presence or absence of multiple alleles or haplotype(s) is to be determined, individual alleles can be detected by any combination of molecular methods (75). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

The discovery cohort used in the GWAS included 1096 Crohn's Disease subjects and 3980 healthy population controls. The replication cohort consisted of 1174 Caucasian CD cases and 357 Caucasian healthy controls; all independent of the cohort in the GWAS. Cases were recruited from the Cedars-Sinai IBD Center and Pediatric IBD department and were diagnosed with CD according to standard clinical, radiological, endoscopic and histological criteria. Controls for the GWAS were obtained from the Cardiovascular Health Study (CHS), a population-based longitudinal study of risk factors for cardiovascular disease and stroke in adults 65 years of age or older, recruited at four field centers (31). 5201 predominantly Caucasian individuals were recruited in 1989-1990 from random samples of Medicare eligibility lists, followed by an additional 687 African-Americans recruited in 1992-1993 (total n=5888). Controls used in the replication study were recruited through the IBD center (unrelated acquaintances and spouses of cases with no personal or family history of IBD or autoimmune disease) or recruited as part of the PARC project, a pharmacogenetic study of statin response (32,33). All cases and controls provided informed consent prior to study participation and following approval of participating centers' institutional review boards.

### Example 2

All genotyping was performed at the Medical Genetics Institute at Cedars-Sinai Medical Center using whole-genome genotyping Infinium technology, following the manufacturer's protocol (Illumina, San Diego, Calif.) (34, 35). Cases were genotyped with either the Illumina Human 610Quad platform or the Illumina Human 317Duo platform. Controls were genotyped with the Illumina 370Duo platform. Samples with genotyping rates >98% were retained in the analysis. In addition, case and control cohorts were both investigated using Identity-By-Descent (Pi hat scores >0.5 as detected in PLINK (36)) in order to identify cryptic relatedness, and related individuals were excluded. Following these QC steps, 1096 CD cases and 3694 controls were included in the study. Single nucleotide polymorphisms (SNPs) were excluded based on the following criteria: test of Hardy-Weinberg Equilibrium p≦10⁻³; SNP failure rate >10%; MAF <5%; and SNPs not found in dbSNP Build 129. SNPs were also examined in order to exclude case/control disparity in missingness (PLINK (36)). 304,825 SNPs that passed QC criteria, and were available in all datasets, were included in the logistic regression association analysis. The 6 SNPs tested in the replication cohort were genotyped using TaqMan™ assay according to the manufacturer's instructions (Applied Biosystems, Foster City, Calif.).

### Example 3

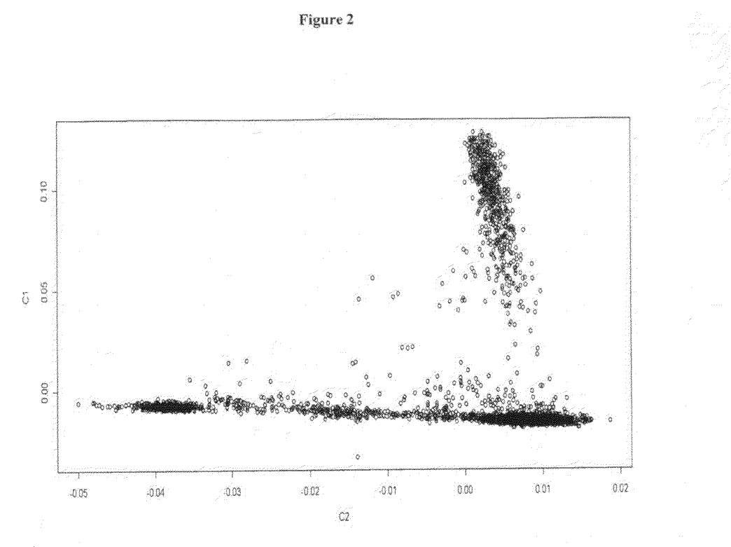

Population structure was detected using Multidimensional Scaling (MDS) (PLINK (36)). In total, 10 principal components (PC) were calculated and plotted for graphical representation of population substructure within the cohort. Subjects with a PC1>0.025 represent African American subjects. To reduce false positive discovery due to population substructure, and the predominantly Caucasian make-up of the cases, these subjects were excluded from downstream analysis. This resulted in 896 CD and 3204 control subjects being carried forward for association testing with the CD phenotype using a logistic regression model in R (FIG. 2). All 10 principal components were carried into association testing as covariates. A logistic regression analysis correcting for population substructure was used to test for association between genotype and phenotype. Self reported ethnicity data was used to confirm the identification of ethnicity based on cluster plots (FIG. 2). The association of the FUT2 SNPs with CD in the independent confirmation cohort was tested using logistic regression (as implemented in R).

### Example 4

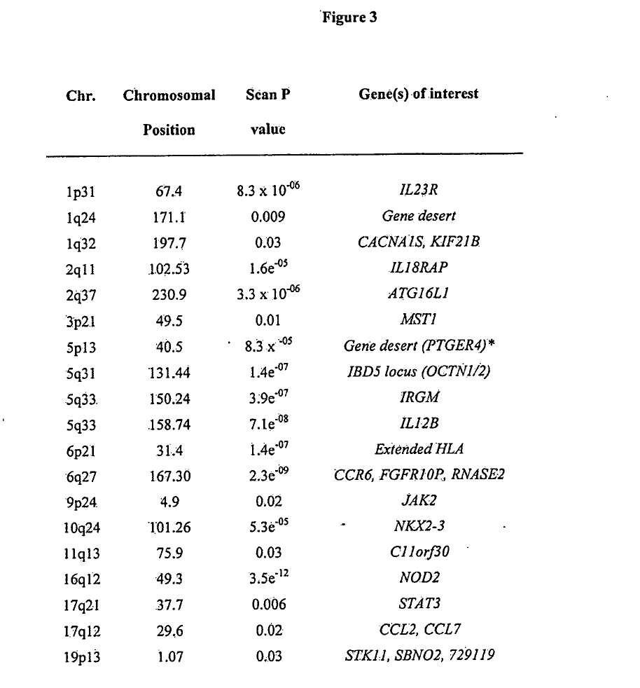

A CD GWAS meta-analysis previously identified or confirmed association with 30 loci and demonstrated nominal association with a further 10 loci (13). The inventors confirmed association (uncorrected p value <0.05 and association with the previously identified risk allele) with 19 of these loci in the inventors' GWAS (FIG. 3) and these loci served as internal controls for the inventors' dataset. Three of these loci were from the nominally replicated list of SNPs (rs4807569, 19p13; rs991804, CCL2, CCL7; rs917997, IL18RAP) from the meta-analysis study, and the data presented in FIG. 3 therefore provide further evidence of their relevance in CD susceptibility. The IL18RAP association has previously been confirmed (37). In this data set the inventors did not demonstrate association (p<0.05) with CD and the other 21 loci identified in the GWAS meta-analysis including 10p11, 10q21, 12q12 (SLC2A13, LRRK2), 1p13 (PTPN22), 18p11 (PSMG2, PTPN2), 17q21 (ORMDL3), 13q14 (CCDC122), 9q32 (TNFSF15), 6p22 (CDKALI), 6q21 (PRDM1), 8q24, 1q23 (ITLN1, CD244,), 6p25(LYRM4), 2p16 (PUS10), 6p25 (SLC22A23), 6q25, 2p23 (GCKR), 7p12, 21q21, 21q22 and 18q11.

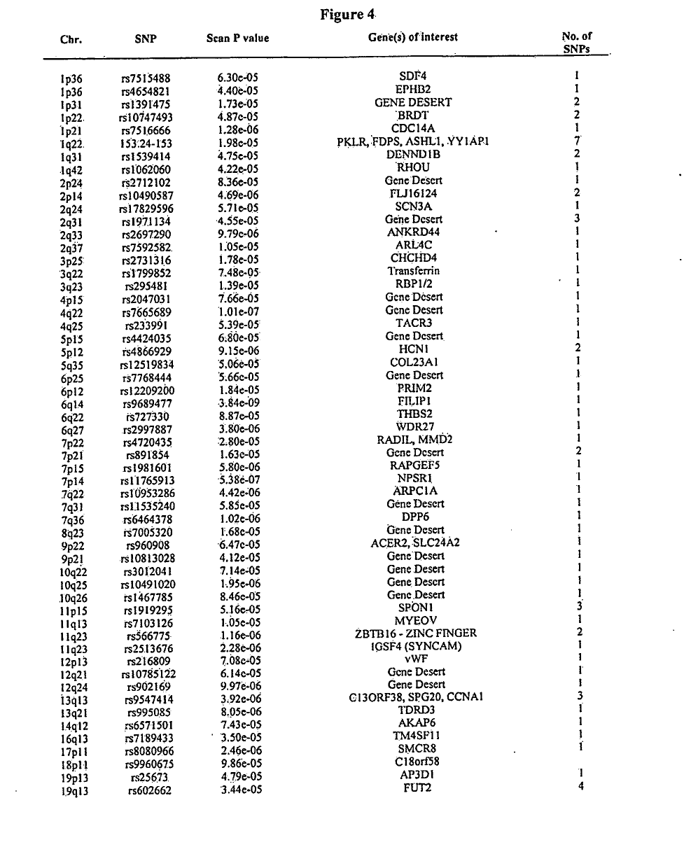

In addition, the inventors identified association between CD and a number of novel loci (FIG. 4). These include genes involved in tight junctions/epithelial integrity (ASHL, ARPC1A), Wnt and JNK1 signaling (RHOU), dendritic cell function (RBP1 and 2), Substance P signaling (TACR3), macrophage development (MMD2), asthma susceptibility (NPSRJ) (38), integrin regulation (ACER2), and NK T cell biology (AP3D1). The inventors also identified two CD associated loci specifically involved in the host-microbial interaction namely SPG20 (endosomal trafficking) and FUT2.

### Example 5

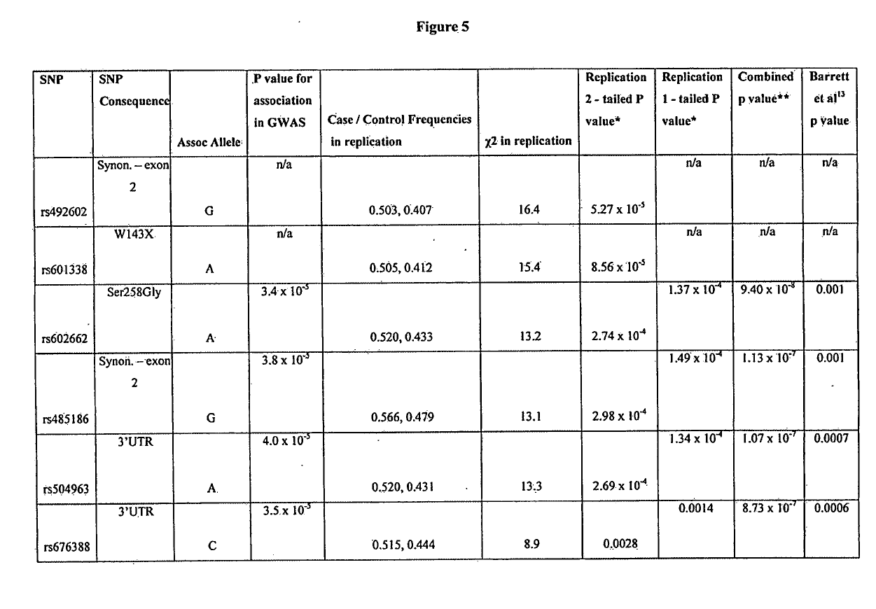

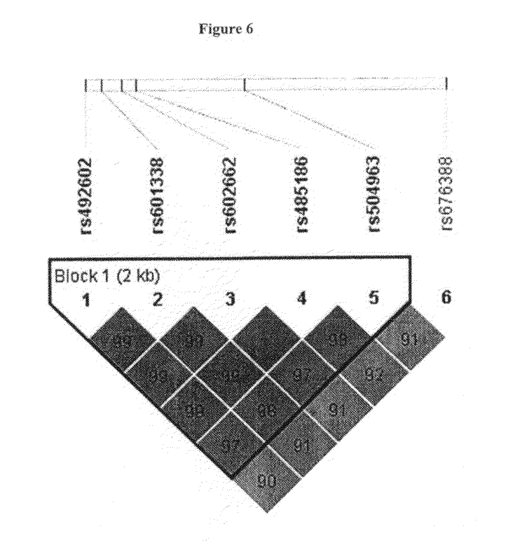

From the novel associations, the inventors first chose FUT2 as the leading gene for independent replication given the inventors' interest in the host-microbial interaction in CD pathogenesis and FUT2's known association with a number of infective processes. Furthermore FUT2 is located under a known peak of linkage for CD on chromosome 19 (39) and there were 4 SNPs with strong association to CD in the inventors' GWAS (FIGS. 5 and 6). In addition to these 4 SNPs (rs504963—3′UTR, rs676388—3′UTR, rs485186—synonymous exon 2 SNP and rs602662—Ser258Gly) identified in the GWAS, the inventors also genotyped rs492602 (synonymous exon 2) and rs601338 (W143X, the common null allele in Caucasians associated with the ABO non-secretory phenotype) in the independent confirmatory cohort. The inventors were able to replicate the initial association with the four SNPs from the discovery cohort, as well as demonstrate association with the additional two SNPs, including the allele for non-secretor status. Further evidence for the association between this locus and CD susceptibility is provided in the CD meta-analysis published by Barrett et al., (13) in which all four of the originally identified SNPs are associated with CD (FIG. 5). The 6 SNPs included in the replication study are in strong linkage disequilibrium (FIG. 6).

### Example 6

In this study the inventors confirmed association with a number of known CD loci and provided further evidence for association to CD with two other loci previously only nominally associated with disease (19p13 and 17q12). The region on 19p13 contains SBNO2 and GPX4(glutathione peroxidase 4). Little is known about SBNO4, while GPX4 is known to protect cells against oxidative damage and may have a regulatory role in leukotriene biosynthesis (40). The 17g12 locus is located in a cytokine gene cluster containing the CCL2, CCL8, CCL11 and CCL7 genes. These genes encode Cys-Cys cytokine genes which are involved in immunoregulatory and inflammatory processes and are therefore attractive candidate genes for CD susceptibility. This locus has previously been implicated in susceptibility to asthma (41) and Mycobacterium susceptibility (42) as well as with HIV progression (43).

Also disclosed herein, the inventors identified novel loci associated with CD, most notably FUT2. The inventors provided independent confirmation for association between FUT2 and CD in both the inventors' own cohort, and in the meta-analysis published by Barrett et al., (13). This cumulative data provides strong evidence of the role of this locus in CD susceptibility. This gene is of particular interest, as it potentially extends knowledge regarding the scope of the host-microbial interaction in CD. Previous genetic associations with CD have highlighted the role of both the innate (11,12,14,15) and the adaptive immune systems' (44,45) interaction with the microbiome. The data presented herein extend this interaction to the mucus layer of the GI tract. FUT2 encodes the secretor type α (1,2) fucosyltransferase (also known as the Se enzyme) that is responsible for regulating the secretion of the ABO antigens in both the digestive mucosa and secretory glands. Approximately 20% of individuals are non-secretors who fail to express ABO antigens in both the GI tract and saliva (17). The prevalence of the non-secretor status (Sc-) is similar between populations (46) although the point mutations that lead to Se- differ. The dominant non-secretor polymorphism in caucasians is the Trp143Ter (W143X) (17) and it is this polymorphism that is implicated in CD in the replication cohort.

Pathogens utilize host cell surface molecules including oligosaccharides (synthesized by glycosyltransferases) for invasion. It is likely that the high prevalence of non-secretor phenotypes in the population occurs due to the absence of particular carbohydrate molecules in the mucosa, and this may have conferred some historical protection to infection as demonstrated with non-secretor status and protection from Helicobacter Pylori infection (18). Lactobacilli, a known commensal bacteria, bind to the precursor glycolipid GA1, implying a role of the GI mucosal glycolipid profile in the adherence of commensal and ‘beneficial’ bacteria, in addition to pathogenic organisms (47). Furthermore Lactobacilli can also displace pathogens such as Clostridium from mucus (48) and inhibit the Shigella-host interaction (49). Commensal bacteria likely induce glycolipid expression, as the fucosylglycolipid FGA1 is found in the small bowel of conventionally bred mice but not in germ-free mice (50). Furthermore FGA1 expression is induced by administration of microbes (51), and FUT2 transcripts in the ileum were induced in germ free mice 48 hours after administration of feces from conventionally bred mice (52). Fut2-null mice do not express the fucosylglycolipid FGA1 in the cecum and colon, whereas normal mice do (50). In the mammalian gut, blocking the CRK and INK pathways inhibits the ability of bacterial colonization to induce fucosyltransferase activity and FUT2 mRNA expression, both of which are hallmarks of the adult mammalian colon (53). Commensal bacteria and probiotics may exert their protective effects through preventing adherence or even displacing pathogenic bacteria, thus emphasizing the potential role of FUT2 and non-secretor status on gastrointestinal bacterial profile (54). It is likely that Se- individuals may thus have a disrupted immunogenic/homeostatic equilibrium that makes them more susceptible to the development of chronic mucosal inflammation, and changes in the microflora of IBD patients have been well-documented (55). There are some data to support this concept, as Fut2 null mice display increased susceptibility to experimental yeast vaginitis and cervical mucins containing Fut2 are partly protected from induced vaginal candidiasis (56).

Although FUT2 is a strong candidate gene for CD susceptibility, given its tissue expression and its influence on the GI bacterial profile, the associations identified in FUT2 may reflect association with other genetic variants at this locus that are in linkage disequilibrium with these SNPs. The inventors therefore explored the LD pattern at this locus using the latest version of HapMap (57) and identified that LD (defined as D′>0.80) extends into neighboring genes, including interesting candidate genes that are also potentially involved in the host-bacterial interaction such as FUT1 (alpha-1-2-fucosyltransferase 1—FUT, genetic variation in pigs is associated with alterations in E. Coli adherence (58)) and RASIPI (RAS interacting protein 1—a RAS effector localized to the Golgi membranes) as well as DBP (D-site of albumin promoter-binding protein) and FGF21 (fibroblast growth factor 21—involved in insulin sensitivity, adipocyte function and growth hormone signalling (59,60)). The inventors believe that FUT2 is an attractive candidate gene at this locus, and have demonstrated association with a variant with a known consequence on gene expression.

In addition, the inventors have identified some novel loci for further investigation, including genes involved in tight junctions, Substance P signaling, macrophage development, dendritic cell function and NK T cell function.

The data disclosed herein provide strong evidence that non-secretor status increases CD susceptibility. The non-secretor variants from other ethnic groups have been well documented, and studies of these variants within the relevant IBD populations will help elucidate the exact role of FUT2 in CD susceptibility. Studies on the effect of FUT2 on clinical and serological phenotype, and particular its role on the microbiome of non-secretor individuals, may help investigators understand further the variation seen in commensal bacteria in individuals with CD, and also further determine those CD patients who might most benefit from probiotic or antibiotic based therapies for prevention and treatment of CD.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

Many modifications and variations of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

1. Kim, S. C. et al., Gastroenterology 128, 891-906 (2005).
2. Rath, H. C. et al., J Clin Invest 98, 945-53 (1996).
3. Rath, H. C. et al., Infect Immun 67, 2969-74 (1999).
4. Rath, H. C. et al., Gastroenterology 116, 310-9 (1999).
5. Gionchetti, P. et al., Gastroenterology 119, 305-9 (2000).
6. Rutgeerts, P. et al., Gastroenterology 108, 1617-21 (1995).
7. Duerr, R. H. et al., Science 314, 1461-3 (2006).
8. Hampe, J. et al., Nat Genet 39, 207-11 (2007).
9. Rioux, J. D. et al., Nat Genet 39, 596-604 (2007).
10. Yamazaki, K. et al., Hum Mol Genet 14, 3499-506 (2005).
11. Hugot, J. P. et al., Nature 411, 599-603 (2001).
12. Ogura, Y. et al., Nature 411, 603-6 (2001).
13. Barrett, J. C. et al., Nat Genet 40, 955-62 (2008).
14. De Jager, P. L. et al., Genes Immun 8, 387-97 (2007).
15. Saruta, M. et al., Inflamm Bowel Dis 15, 321-7 (2009).
16. Mow, W. S. et al., Gastroenterology 126, 414-24 (2004).
17. Kelly, R. J. et al., J Biol Chem 270, 4640-9 (1995).
18. Ikehara, Y. et al., Cancer Epidemiol Biomarkers Prey 10, 971-7 (2001).
19. Marionneau, S. et al., J Infect Dis 192, 1071-7 (2005).
20. Thorven, M. et al., J Virol 79, 15351-5 (2005).
21. Carlsson, B. et al., PLoS One 4, c5593 (2009).
22. Kindberg, E. et al., AIDS 20, 685-9 (2006).
23. Hazra, A. et al., Nat Genet 40, 1160-2 (2008).
24. Thom, S. M. et al., FEMS Microbiol Immunol 1, 401-5 (1989).
25. Aly, F. Z. et al., Epidemiol Infect 106, 355-63 (1991).
26. Haverkorn, M. J. & Goslings, W. R. Am J Hum Genet 21, 360-75 (1969).
27. Kinane, D. F. et al. , Br Med J (Clin Res Ed) 285, 7-9 (1982).
28. Chaudhuri, A. & DasAdhikary, C.R. Trans R Soc Trop Med Hyg 72, 664-5 (1978).
29. Blackwell, C. C. et al., Lancet 2, 284-5 (1986).
30. Blackwell, C. C. et al., Lancet 2, 687 (1986).
31. Fried, L. P. et al., Ann Epidemiol 1, 263-76 (1991).
32. Krauss, R. M. et al., Circulation 117, 1537-44 (2008).
33. Simon, J. A. et al., Am J Cardiol 97, 843-50 (2006).
34. Gunderson, K. L. et al., Nat Genet 37, 549-54 (2005).
35. Gunderson, K. L. et al., Methods Enzymol 410, 359-76 (2006).
36. Purcell, S. et al., Am J Hum Genet 81, 559-75 (2007).
37. Zhernakova, A. et al., Am J Hum Genet 82, 1202-10 (2008).
38. Laitinen, T. et al., Science 304, 300-4 (2004).
39. van Heel, D. A. et al., Hum Mol Genet 13, 763-70 (2004).
40. Villette, S. et al., Blood Cells Mol Dis 29, 174-8 (2002).
41. Batra, J. et al., J Med Genet 44, 397-403 (2007).
42. Thye, T. et al., Hum Mol Genet 18, 381-8 (2009).
43. Modi, W. S. et al., AIDS 17, 2357-65 (2003).
44. Shen, C. et al., Inflamm Bowel Dis 14, 1641-51 (2008).
45. Duchmann, R. et al., Eur J Immunol 26, 934-8 (1996).
46. Pang, H. et al., Ann Hum Genet 65, 429-37 (2001).
47. Yamamoto, K. et al., Biochem Biophys Res Commun 228, 148-52 (1996).
48. Lee, Y. J. et al., Int J Antimicrob Agents 21, 340-6 (2003).
49. Moorthy, G. et al., Dig Liver Dis (2009).
50. Iwamori, M. & Domino, S. E. Biochem J 380, 75-81 (2004).
51. Lin, B. et al., Arch Biochem Biophys 388, 207-15 (2001).
52. Lin, P. H. et al., Am Surg 66, 627-30 (2000).
53. Meng, D. et al., Am J Physiol Gastrointest Liver Physiol 293, G780-7 (2007).
54. Collado, M. C. et al., Lett Appl Microbiol 45, 454-60 (2007).
55. Swidsinski, A. et al., Inflamm Bowel Dis 14, 147-61 (2008).
56. Hurd, E. A. & Domino, S. E., Infect Immun 72, 4279-81 (2004).
57. Frazer, K. A. et al., Nature 449, 851-61 (2007).
58. Meijerink, E. et al., Immunogenetics 52, 129-36 (2000).
59. Berglund, E. D. et al., Endocrinology (2009).
60. Inagaki, T. et al., Cell Metab 8, 77-83 (2008).
61. Podolsky, et al., N Engl J Med 347, 417 (2002).
62. Loftus, et al., Gastroenterology 126, 1504 (2004).
63. Vermeire, et al., Genes Immun 6, 637 (2005).
64. Hugot, et al., Nature 411, 599 (2001).
65. Rioux, et al., Nat Genet 29, 223 (2001).
66. Peltekova, et al., Nat Genet 36, 471 (2004).
67. Jarcho et al. in Dracopoli et al., Current Protocols in Human Genetics pages 2.7.1-2.7.5, John Wiley & Sons, New York.
68. Kutyavin, et al., Nucleic Acids Research 28:655-661 (2000).
69. Mullis, et al. (Eds.), The Polymerase Chain Reaction, Birkhauser, Boston, (1994).
70. Delwart, et al., Science 262:1257-1261 (1993).
71. White, et al., Genomics 12:301-306 (1992).
72. Hayashi, K., Methods Applic. 1:34-38 (1991).
73. Innis, et al.,(Ed.), PCR Protocols, San Diego: Academic Press, Inc. (1990).
74. Winter, et al., Proc. Natl. Acad. Sci. 82:7575-7579 (1985).
75. Birren, et al. (Eds.) Genome Analysis: A Laboratory Manual Volume 1 (Analyzing DNA) New York, Cold Spring Harbor Laboratory Press (1997).

1. A method of diagnosing susceptibility to Crohn's disease in an individual, comprising:
obtaining a sample from the individual;
assaying the sample to determine the presence or absence of a risk variant at the FUT2 genetic locus; and
diagnosing susceptibility to Crohn's disease in the individual based on the presence of the risk variant at the FUT2 genetic locus.

2. The method according to claim 1, wherein the risk variant is selected from the group consisting of rs602662, rs676388, rs485186, and rs504963.

3. The method of claim 1, wherein assaying the sample comprises genotyping for one or more single nucleotide polymorphisms.

4. The method according to claim 1, wherein the sample is whole blood, plasma, serum, saliva, cheek swab, urine, or stool.

5. A method of prognosing Crohn's disease in an individual, comprising:
obtaining a sample from the individual;
assaying the sample for the presence or absence of one or more genetic risk variants; and
prognosing an aggressive form of Crohn's disease based on the presence of one or more risk variants at the FUT2 genetic locus.

6. The method according to claim 5, wherein the risk variant is selected from the group consisting of rs602662, rs676388, rs485186, and rs504963.

7. The method of claim 5, wherein assaying the sample comprises genotyping for one or more single nucleotide polymorphisms.

8. The method according to claim 5, wherein the sample is whole blood, plasma, serum, saliva, cheek swab, urine, or stool.

9. A method of treating an individual for Crohn's disease, comprising:
prognosing an aggressive form of Crohn's disease in the individual based on the presence of one or more risk variants at the FUT2 genetic locus; and
treating the individual,
wherein the one or more risk variants are selected from rs602662, rs676388, rs485186, and rs504963.

10. The method of claim 9, wherein assaying the sample comprises genotyping for one or more single nucleotide polymorphisms.

11. The method according to claim 9, wherein the sample is whole blood, plasma, serum, saliva, cheek swab, urine, or stool.

12. A method of determining a high probability of developing Crohn's disease in an individual, relative to a healthy subject, comprising:
obtaining a sample from the individual;
assaying the sample to determine the presence or absence of one or more risk variants at the FUT2 genetic locus; and
diagnosing a high probability of developing Crohn's disease in the individual, relative to a healthy subject, based upon the presence of the one or more risk variants at the FUT2 genetic locus.

13. The method according to claim 12, wherein the one or more risk variants are selected from the group consisting of rs602662, rs676388, rs485186, and rs504963.

14. The method of claim 12, wherein assaying the sample comprises genotyping for one or more single nucleotide polymorphisms.

15. The method according to claim 12, wherein the sample is whole blood, plasma, serum, saliva, cheek swab, urine, or stool.
